(19) United States
(12) Patent Application Publication
(10) Pub. No.: US 2021/0265045 A1
(43) Pub. Date: Aug. 26, 2021

(54) System and method for using unique identifying data for precautionary determination to send notifications to a user to reduce potential negative health outcomes related to implantable devices and recall, adverse event data

(71) Applicants: Jeremy Michael Elias, Kansas City, MO (US); Caitlin Marie Elias, Kansas City, MO (US)

(72) Inventors: Jeremy Michael Elias, Kansas City, MO (US); Caitlin Marie Elias, Kansas City, MO (US)

(21) Appl. No.: 16/373,619

(22) Filed: Apr. 2, 2019

(51) Int. Cl. G16H 40/20; G06F 16/245; G06F 16/25; G06F 9/54; G16H 10/60; G06Q 30/00; G16H 50/70; G16H 30/20; G16H 40/67; H04L 12/58

(52) U.S. Cl. CPC G16H 40/20; G06F 16/245; G06F 16/252; G06F 9/541; G16H 10/60; G06Q 50/26; G16H 50/70; G16H 30/20; G16H 40/67; H04L 51/24; G06Q 30/014

(57) ABSTRACT

One method involves pulling, or receiving, a list of recalled device data. Subsequently, the recalled device data is compared to information in a user's TrackMy health record. Based on this comparison, a determination is made as to whether one or more matches exist between any of the recalled devices in the list and the TrackMy health record information, the match relating to the potential for a negative health outcome if the user is not notified of this. If a match exists, a response is outputted relating to each match and a notification is sent to the user's contact information stored TrackMy health record.

### CROSS-REFERENCE TO RELATED APPLICATIONS

This application claims the benefit of U.S. Provisional Application No. 62656975 filed Apr. 10, 2018.

### BACKGROUND INFORMATION

In the health care industry, there is a large desire to increase patient safety through the use of data-driven solutions. Consumer-driven health care and interoperability, along with full access to an individual's health record is becoming a vital need and reality to lead to better care for patients. It is the right of the patient to have full access to their data. Our invention supports these factors, and allows a user/patient to access their data, and make this data actionable to increase their safety.

To denote some reasoning that led to our invention, and the need for this patent protection
Recent studies show there has been an increase of device recalls year over year (citing a report from the FDA/CDRH—97% from FY 2003 to 2012 for example)
   Recalls continue to happen at an alarming rate year over year. There is a lot of good that comes from implantable devices, yet a lot of harm as well. This invention helps increase safety and hold accountable appropriate parties should complications arise.
Inefficient Recall Strategy for Device Manufacturers
   The average number of days from firm awareness to recall posting ranged between 233.7-256.6 days. Our patient outcome tracker invention immediately notifies Health systems/EMR vendors and patients when a recall happens. We have created a patient list in order for the Health systems to be able to directly communicate with the patients of a device recall and plan next steps (along with allowing the patients next steps via the features within the invention/application).
Lack of Patient and Public Health Awareness around implant tracking
Patient Fatalities related to defective implantable devices
   This application supports the Recall Process Improvement that was kicked off CDRH (Center for Devices and Radiological Health) and the FDA (Food and Drug Administration) in 2010
Per past articles and research there are over ˜2000 device recalls in the US Annually
   Due to these device recalls, they cause an undefined amount of issues with a given patient (our application can start to better track this data, and push public health to the forefront especially as ˜10,000 baby boomers retire daily (reference Washington Post article from 2016) as of 2016, and a lot of this generation have device implants for varying reasons). These issues comprise of device malfunctions, poor design of devices and can be cut down upon use of our invention. Being built to the Smart on FHIR specifications, this invention can be scalable to pull data from EMRs and be able to do a lot of things with it in conjunction with the CMS (Centers for Medicare/Medicaid) push to capture this data. Better overall communication plans of device recalls and notification of patients of high-risk items
In today's marketplace, the leading comparable approach to this invention is mass advertising through outlets like television networks. Attorneys will discover a recall and start to run mass advertisements through larger TV networks trying to target patients that have these recalled devices.
At times a person will be tech-savvy enough to sign up for email alerts, or hear about a recall through the news as noted above. Herein lies the issue, if this person does not actually know what exact device they (or a loved one/family member etc) has there is no way to positively identify a given recall is on the actual device they have implanted.
Someone in theory (either a user or a healthcare worked), could also manually monitor the FDA website for recalls. But even if this information is available, the person/worker must still reference/consult another set of information- specifically a list derived from a medical record or elsewhere that would need to be at a detailed enough level to compare to any FDA posted recall information. This effort of looking up or “cross-checking” is subject to human error, very time consuming, thus negatively impacting the efficiency of a health care process that can impact the livelihood of an individual. In summary, cross-checking becomes increasingly complex to ensure a positive identity of a person, to a device, to a recall.
These noted processes are not effective, and we are doing patients/users a dis-service through this process. Our invention augments this, and allows for direct communication with implant patients/users to increase patient safety.

### SUMMARY OF THE INVENTION

System and method are implemented for using unique identifying data for precautionary determination to send notifications to a user to reduce potential negative health outcomes related to implantable devices and recall, adverse event data. In this way, incidents of recalls/adverse events are tracked, and patient safety increases along with positive clinical outcomes.

In one perspective of the invention, data is stored by user manually using a User Interface (TrackMy Implants Application) to search and save an Implantable device.

This method includes the user using the TrackMy user interface to search through a search bar that is connected to a general device database (GUDID API endpoint) for a given device, and once they find the device they are looking for, click save and this is stored in the TrackMy control server/database.

In one perspective of the invention, data is stored by capturing of unique device data through the use of Smart on FHIR integration with electronic medical records. This is further defined in the detail section, yet herein this process is as follows—user clicks a button on a webpage to launch a given EMR Authentication API (this is normally a patient portal login or the like; also some EMRs use Google Identity verification), the consents to share medical data with TrackMy, and FHIR data for the given positively identified user loads on the webpage. FHIR device data, and any necessary corresponding data is then stored on the TrackMy control server/database for tracking. This populates the device-person table.

In one perspective of the invention, data is stored by the direct interface of unique device data to server (TrackMy) through an HL7 established interface with another healthcare related technology system. This data would be positively identified through a unique user identifier, and data stored on the TrackMy control server/database for tracking. This populates the device-person table.

In one perspective of the invention, data is stored by the manual upload/data entry into the device-person table by an individual or through an automated scanning process. In some scenarios, agreements will be worked out between data providers and TrackMy to manually enter the user device data directly into TrackMy control server/database by an individual or through an automated scanning process. This populates the device-person table.

### BRIEF DESCRIPTION OF THE DRAWINGS

In referring to the drawings,

FIG. 1 provides a block diagram of the operation of the invention;

FIG. 2 provides a block diagram of the invention during manual usage; and,

FIG. 3 provides a block diagram of the invention integrating with electronic medical records.

The same reference numerals refer to the same parts throughout the various figures.

### DETAILED DESCRIPTION

Our invention is an application and website that notifies patients of applicable active and inactive recalls impacting their implantable devices. Securely collate data around patients, their implanted devices, and any recalls and adverse effects in order to offer solicitations that the user would find relevant

Core Features: 1.) Application and website (hereafter referred to as A&W) to meet HIPAA certification guidelines. 2.) A&W shares user database and content. Adding a device to a user profile in either A or W synchronizes across both and any future platforms. 3.) A&W permits access to iOS and Android technology platforms as well as common web browsers.

Data Population Processes
The user manually using a User Interface (TrackMy Implants Application) to search and save an Implantable device or
The capturing of unique device data through the use of Smart on FHIR integration with electronic medical records or
The direct interface of unique device data to server (TrackMy) through an HL7 established interface with another healthcare related technology system or
The manual upload/data entry into the device-person table by an individual or through an automated scanning process

Detailed Data Population Processes

Software application details/process steps for user manually using TrackMy User Interface to populate the device-person table:
1.) Software application allows Device search from the FDA “DeviceUDI” API dataset. The API is being called from the FDA through the mobile application using one of three search types (see Device Search screenshot in Visual tab section):

App Search field name App Display field name DeviceUDI API field name Device ID Primary DI Number “results_identifiers_id” Name Brand Name “results_brand_name” Company Name “results_company_name”
2.) In addition to the App Display field names shown above, two additional fields are displayed to the user. App Display field name DeviceUDI API field name Regulation Number “results_product_codes_openfda_regulation_number” Device Description “results_device_description ”
3.) Once the user has saved the appropriate devices to their profile, the devices are checked against the “Device-Recall” Parsed out table nightly (the recall check process is run every 24 hours) using the Device Identifier value. The Device-Recall table is created through TrackMy triggering a pull from the OpenFDA Device Enforcement Report API, and we then parse out the “code_info” field to pull out Device Identifiers through an algorithm and rules we have created.
4. ) If a recall match is found, then the user receives a notification through the application. Along with a text message (if the user saved their contact information), and a phone message automated call notification as well
In addition to using the recall and device data, we are leveraging Device Adverse Event data to derive the needs for a notification, additional details on adverse events are as follows
   Detailed description of Adverse Event usage—Adverse Events impact devices that have not yet been recalled. They indicate that something has happened involving a particular device, but the device itself has not yet been identified as causing the incident. This information may be available months or years in advance of a recall. As such, sharing of such information with patients must be handled lightly. Methods of joining “DeviceUDI” to “Adverse Event” are ‘results_device_catalog_number’ or ‘results_device_model_number’

The capturing of unique device data through the use of Smart on FHIR integration with electronic medical records
1.) To assist users in building their device profiles, the use of Smart on FHIR APIs are leveraged. This allows the user to connect to their EHR records and download their implantable device data into TMS database (this is further defined in Model C below and in the drawings for Model C).
2.) In addition, we will be offering patient education tied to the user's device list (patient education content company to be named TBD; ex- UpToDate)
3.) Users should be contacted by email as well as push notification along with phone messages
4.) Load article links in app to push to users. Linking WordPress blog so that as we post to the blog, link to user.
This is further detailed in the following sections.

The direct interface of unique device data to server (TrackMy) through an HL7 established interface with another healthcare related technology system or
1. ) An established relationship would be built between TrackMy and a given client (i.e.—Hospital); a unique identifying data element—i.e.—SSN, Patient ID, etc., would be exchanged between TrackMy and Client to positively identify a given patient.
2.) Once this is done, Client can send patient data (once patient has went in and consented) to TrackMy to be loaded on the device-person table.

The manual upload/data entry into the device-person table by an individual or through an automated scanning process

1.) For this process/data upload, this assumes patient consent has been established upon account creation.

2.) Patient/User allows TrackMy to work with Surgeon/Provider to upload Device Identifier data into database and load on the device-person table

TrackMy Implants Architecture

As currently notated, there are three models for the architecture.

Model A, as shown in FIG. 1 is as follows

FIG. 1—Model A—Manual—TrackMy Technology Patent is being submitted to cover all of these model flows, as we reserve the right to leverage enhancements to technology for optimal performance for our end-user community as well as there is more than one way to get data into the TrackMy database/servers.

FIG. 1 utilizes the following reference characters shown in index form:

100 TrackMy Implants Website

110 TrackMy Implants App

120 Open FDA API Recall UDI

130 Open FDA API Device UDI

140 TrackMy Database

10 line between 100 and 120, call recall API by Device Details, recall returned if matched

11 line between 100 and 130, recall process

40 line between 100 and 140, flow

41 line between 100 and 110, like user experience

42 line between 110 and 120, user search call to API, user search results returned from API, bidirectional dataflow

43 line between 110 and 130, user search call to API, user search results returned from API, bidirectional dataflow

44 line between 110 and 140, flow

Flow
User has downloaded TrackMy Implants technology or logged in via Web App
User Signs in with Email address, Single Sign On with Facebook, Google, or Creates a New Account
User clicks search button (see on app visual tab), enters data to search (only can search what API allows today (leads to Model B)) and it evokes the API call to Device UDI
User sees information on device (see on app visual tab) and saves device
Recall API call runs nightly and triggers on saved device information
If Recall Match takes place, push notification and text message and phone call (if have data) is sent to user (see recall visual)
TrackMy Database
   User Data Saved to Database; Saved Devices Show to User from Database; Alerts saved to Database once triggered
   Every 24 hours, we look for new data by repulling the API data, normalizing it, and reloading into our Database Schema
   The Following OpenFDA APIs we are pulling into our Database, normalizing and allowing for a more robust user search/recall process
      OpenFDA APIs we are using, are all that exist today at—https://open.fda.gov/device/
      https://open.fda.gov/device/event/
      https://open.fda.gov/device/classification/
      https://open.fda.gov/device/510k/
      https://open.fda.gov/device/pma/
      https://open.fda.gov/device/registrationlisting/
      https://open.fda.gov/device/recall/
      https://open.fda.gov/device/enforcement/
      https://open.fda.gov/device/udi/

The application and website hit the OpenFDA APIs on demand, store retrieved information in a user profile database shared across all A&W platforms. As noted above, the profile would also eventually include information retrieved from the user's Electronic Health Records (EHR), which would be used to trigger the Recall API. The Recall API is called at least nightly for all user devices in the database. The Device API will continue to be called on demand by manual search. Exception reports would be enabled on a weekly or daily basis to ensure that all devices in the database can be matched to records from the Device API (ensuring data integrity for items added through EHR integration).

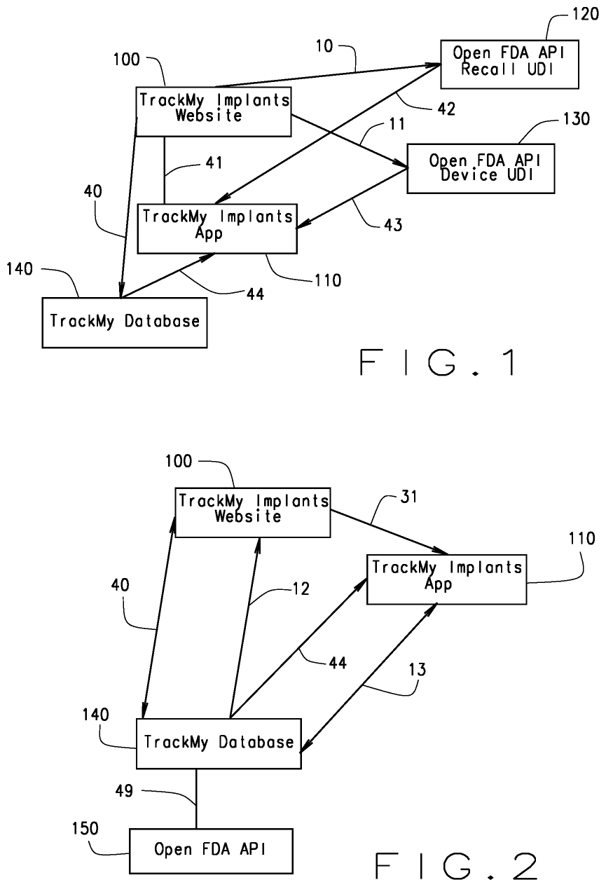

Model B, as shown in FIG. 2 is as follows

FIG. 2 utilizes the following reference characters shown in index form:

100 TrackMy Implants Website

110 TrackMy Implants App

140 TrackMy Database

150 Open FDA API

12 line between 100 and 140, call database for recall by device details

13 line between 110 and 140, call database for recall by device details

40 line between 100 and 140, flow

41 line between 100 and 110, like user experience

44 line between 110 and 140, user search call to API, user search results returned from API, bidirectional dataflow

49 line between 140 and 150, flow

Flow
User has downloaded TrackMy Implants technology or logged in via Web Application
User Signs in with Email address, Single Sign On with Facebook, Google, or Creates a New Account
User clicks search button (see on app visual tab), enters data to search (this search is controlled by TrackMy and is from our database, where we can provide an improved user search)
User sees information on device (see on app visual tab) and saves device
Recall Match Process call runs nightly and triggers on saved device information
If Recall Match takes place, push notification and text message and phone call (if have data) is sent to user (see recall visual)TrackMy Database
   User Data Saved to Database; Saved Devices Show to User from Database; Alerts saved to Database once triggered
   Every 24 hours, we look for new data by repulling the API data, normalizing it, and reloading into our Database Schema
   The Following OpenFDA APIs we are pulling into our Database, normalizing and allowing for a more robust user search/recall process
      OpenFDA APIs we are using, are all that exist today at—https://open.fda.gov/device/
      https://open.fda/gov/device/event
      https://open.fda.gov/device/510k)
      https://open.fda.gov/device/classification/
      https://open.fda.gov/device/510k/
      https://open.fda.gov/device/pma/
      https://open.fda.gov/device/registrationlisting/
      https://open.fda.gov/device/recall/
      https://open.fda.gov/device/enforcement/
      https://open.fda.gov/device/udi/

Directing all query calls to a hosted database containing all available API content from the FDA. The FDA makes these datafiles available on a daily basis, thus giving TrackMy more control over the data and reduce our API calls to the FDA by regularly downloading the files. This allows flexibility to add additional API datasets should they become available (potentially in the Medicare Claims space).

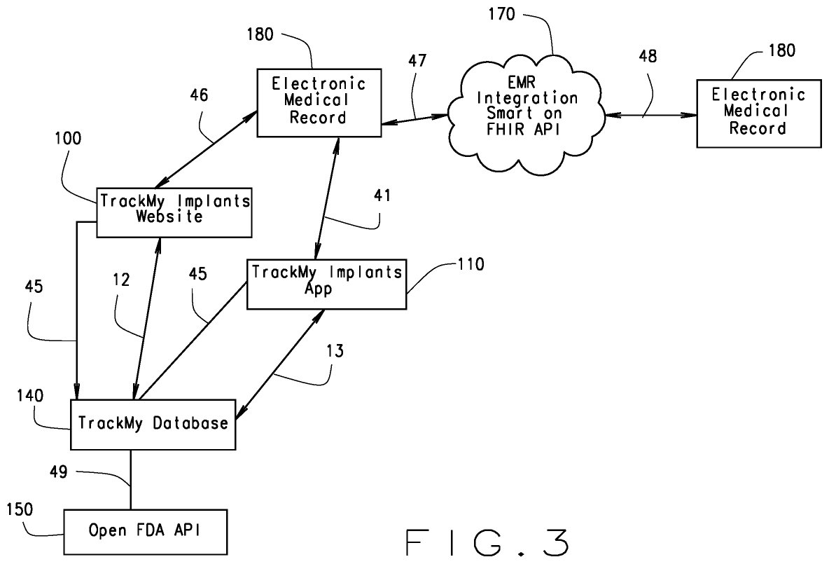

Model C, as shown in FIG. 3 is as follows

FIG. 3 utilizes the following reference characters shown in index form:

100 TrackMy Implants Website

110 TrackMy Implants App

140 TrackMy Database

150 Open FDA API

160 Authentication

170 EMR Integration Smart on FHIR API

180 Electronic Medical Record

12 line between 100 and 140, call database for recall by device details, recall returned if match

13 line between 110 and 140, call database for recall by device details, recall returned if match

41 line between 110 and 160, flow

45 line between 100 and 140, augmented device details, search

46 line between 100 and 160, bidirectional flow

47 line between 160 and 170, bidirectional flow

48 line between 170 and 180, bidirectional flow

49 line between 140 and 150, flow

Flow
User has downloaded TrackMy Implants technology or logged in via Web Application
User Signs in with Email address, Single Sign On with Facebook, Google, or Creates a a New Account
User Authenticates through Identity Provider of Healthcare Organization (ie—Patient Portal Credentials; Oauth Process)
User is presented with Available Medical Device Data from Electronic Health Record Vendor (ie—Allscripts; dependent who is connected to our business)
User Accepts Medical Device Data and Signs Patient Consent to save data into TrackMy Database
Recall Match Process call runs nightly and triggers on saved device information
If Recall Match takes place, push notification and text message and phone call (if have data) is sent to user (see recall visual)
TrackMy Database
   User Data Saved to Database; Saved Devices Show to User from Database; Alerts saved to Database once triggered
   Every 24 hours, we look for new data by repulling the API data, normalizing it, and reloading into our Database Schema
   The Following OpenFDA APIs we are pulling into our Database, normalizing and allowing for a more robust user search/recall process
      OpenFDA APIs we are using, are all that exist today at—https://open.fda.gov/device/
      https://open.fda.gov/device/event/
      https://open./fda./gov/device/classification/
      https://open.fda.gov/device/510k/
      https://open.fda.gov/device/pma/
      https://open.fda.gov/device/registrationlisting/
      https://open.fda./gov/device/recall/
      https://open.fda.gov/device/enforcement/
      https://open.fda.gov/device/udi/

Replaces the Manual search and save by adding in the Smart on FHIR Integration with varying Electronic Medical Records (ie—Allscripts). There still is an augmented search tied into the FHIR/EMR Integration should relevant data not be brought fully in via the EMR Integration. There is an authentication process the user has to complete using OAuth and authenticating through the Identity Provider (in this case an example is the Hospital Systems/organizations Patient Portal). The recall process remains intact/the same for all Models. This technology is not limited to a particular Electronic Medical Record Vendor (ie Allscripts), our technology is vendor agnostic and we will determine through agreements between various EMR vendors and

TrackMy; based on business needs and ongoing partnerships etc.

Any architectural model used must accommodate three stakeholder groups. 1) The patients who log into the application are assured they maintain control of their data. 2.) Admin ability to monitor changes in data and resolve exceptions where stored user devices cannot be reconciled against the DeviceUDI dataset. 3.) Third Party options to pay for leads to patients impacted by Recalls (initially) and Adverse Effects (later). Users may not be contacted without providing permission first; at minimum this will require an agreement checkbox or opt out capability. Further discussion will be required to determine the nature of these solicitations, but development has proceeded with this objective in mind.

Examples of how it will be used:
Patients can/will use technology to track and receive alerts of recalled medical devices
Patients can/will receive electronic patient education through technology
Attorney usage—through patient consent be able to communicate to patients with potential recalls
Device Manufacturers—partner with TrackMy technology to improve their recall strategy
Surgery Centers/Physicians—use technology to notify their patient population of medical device recalls/adverse events based on what devices they have FDA/CDRH—use technology to better enforce recall strategy and track what patients have been notified—leading to an increase public health
This software will increase patient safety and lead to an overall improved public health
This software has real potential save a patient's life through real-time notifications This software allows a user to better manage their overall health through better information
This software educates a user of a device recall they have on a saved device

What is claimed is:

1. A method in a computing system for preemptive determination of the potential to send a notification to reduce potential negative health outcomes related to implantable devices and recall, adverse event data to a person having an electronic health record, the method comprising the steps of:
accessing device-person tables that maintain specific sets of unique device identifier data including device identifier, lot number, serial number, expiration date;
accessing extracted device-recall tables that maintain specific sets of unique device recall data including device identifier, lot number, serial number, expiration data, recall data, reason for recall;
access extracted device-adverse_event tables that maintain specific sets of unique device adverse event data including device identifier, lot number, serial number, expiration date;
employing a control server to compare the device-person table data against device-recall data;
determining that at least one match exists between any of the selected devices included in the device-person, and device-recall tables, wherein at least one match indicates the potential for a negative health outcome if the user is not notified;
determining that at least one match exists between any of the selected devices included in the device-person, and device-adverse_event tables, wherein at least one match indicates the potential for a negative health outcome if the user is not notified;
sending a notification to a user following determination of a match existing using a user's demographic contact information stored in person table and sending said notification to medical staff of the user;
storing said notification in said device-match table for future reference; and,
creating a user to device list wherein upon a recall occurring the present invention tracks which users previously received said notification and sending this list of previously informed user to medical staff of them.

2. The method of claim 1, wherein the accessing device-person tables include:
data population of this table through one of the user manually using a User Interface (TrackMy Implants Application) to search and save an Implantable device, the capturing of unique device data through the use of Smart on FHIR integration with electronic medical records, the direct interface of unique device data to server (TrackMy) through an HL7 established interface with another healthcare related technology system, and the manual upload/data entry into the device-person table by an individual or through an automated scanning process.

3. The method of claim 2, wherein the user manually uses the TrackMy Implants User Interface to populate the device-person table consists of using a search box that is connected to a central database for devices that get manufactured and contains corresponding identifying information for an individual device.

4. The method of claim 2, wherein the capturing of unique device data through the use of Smart on FHIR integration with electronic medical records to populate the device-person table consists of a user clicking a button that calls a FHIR API endpoint for either an electronic medical record or a patient portal system.

5. The method of claim 2, wherein the direct interface of unique device data and user demographic data to server (TrackMy) through an interface with another healthcare related technology system consists of the direct interface sending of unique implantable device data and this data to be matched to unique user demographic data, the server then saves this data to the device-person table used for ongoing tracking

6. The method of claim 1, wherein the data population of the extracted device-recall table consists of a making a direct API call a central database that stores devices that have been recalled and corresponding recall details.

7. The method of claim 1, wherein the data population of the extracted device-adverse event table consists of a making a direct API call a central database that stores devices that have had adverse events and corresponding adverse event details.

8. The method of claim 1, wherein the potential negative health outcomes include:
death wherein a recall occurs without dissemination to a user who perishes;
life-changing injury wherein a recall occurs with delayed dissemination to a user who endures injury until acting upon the recall; and,
injury wherein a recall occurs from metallosis.

9. The method of claim 1, wherein determining that at least one match exists between any of the selected devices of the device-person table and the device-recall tables further comprises:
matching at least one of device identifier, lot number, serial number, expiration date; and,
verifying at least a minimum match upon device identifier and wherein no match on device identifier occurs the present invention does not notify a user and wherein a match on device identifier occurs the present invention generates an action on the control server to send a notification to the user said notification informing the user to seek immediate medical attention.

10. The method of claim 1, wherein determining that at least one match exists between any of the selected devices included in the device-person table and the device-adverse_event table further comprises:
matching at least one of device identifier, lot number, serial number, expiration date; and,
verifying at least a minimum match on device identifier and wherein no match on device identifier occurs the present invention does not notify a user and wherein a match on device identifier occurs the present invention generates an action on the control server to send a notification to the user said notification informing the user to seek immediate medical attention.

11. The method of claim 1, wherein the server sending out a notification on a positive matched recalled device utilizes user demographic data stored on the person table and care team information for that user stored on the person table;
the present invention generates an electronic mail sent to the email address of the user from the person table and generates a text message and a phone call message sent to the phone number stored on the Person table.

12. The method of claim 1, wherein the storing of the notification on the device-match table comprises saving this notification timestamp to this table.
